(19) United States
(12) Patent Application Publication
(10) Pub. No.: US 2020/0054341 A1
(43) Pub. Date: Feb. 20, 2020

(54) DEVICES AND METHODS FOR ISOLATING A TREATMENT REGION IN THE BODY FROM OTHER REGIONS

(71) Applicant: CSA Medical, Inc., Lexington, MA (US)

(72) Inventor: Thomas I. Mulcahey, Bedford, MA (US)

(21) Appl. No.: 16/539,105

(22) Filed: Aug. 13, 2019

Related U.S. Application Data
(60) Provisional application No. 62/718,567, filed on Aug. 14, 2018.

(51) Int. Cl. A61B 17/12

(52) U.S. Cl. CPC A61B 17/1204; A61B 2017/00862; A61B 17/12159; A61B 17/12099

(57) ABSTRACT

The present disclosure relates generally to the field of medical devices and procedures for isolating a treatment region in the body from other regions, such as a treatment region in the digestive tract from other regions of the digestive tract. In particular, the present disclosure relates to devices and methods for performing a treatment within the upper gastrointestinal tract, esophagus, gastroesophageal junction, and/or stomach that utilizes a barrier member to isolate a proximal region of the digestive tract from a distal region of the digestive tract such that fluids introduced during the treatment do not substantially communicate between the regions.

### PRIORITY

This application claims the benefit of priority under 35 USC § 119 to U.S. Provisional Patent Application Ser. No. 62/718,567, filed Aug. 14, 2018, which is incorporated by reference herein in its entirety and for all purposes.

### FIELD

The present disclosure relates generally to the field of medical devices and procedures for isolating a treatment region in the body from other regions, such as a treatment region in the digestive tract from other regions of the digestive tract. In particular, the present disclosure relates to devices and methods for performing a treatment within the upper gastrointestinal tract, esophagus, gastroesophageal junction, and/or stomach that utilizes a barrier member to isolate a proximal region of the digestive tract from a distal region of the digestive tract such that fluids introduced during the treatment do not substantially communicate between the regions.

### BACKGROUND

In various medical treatments within the vasculature and other lumens of the body, for example, the upper gastrointestinal tract, esophagus, gastroesophageal junction, stomach, lower gastrointestinal tract, small intestines, large intestines, respiratory tract, trachea, and bronchia, it may be desired for the treatment region to be isolated, such that the treatment applied is delivered to the treatment region only and, additionally, any byproducts of the medical treatment, such as cryogen gas from cryotherapy, may be kept from drifting to undesired regions of the body.

As an example, in cryotherapy, cryoablation is a surgical procedure in which diseased, damaged or otherwise undesirable tissue (collectively referred to herein as “target tissue” and/or “treatment region”) may be destroyed by local delivery of a cryogen spray. These systems along with other cryotherapy systems are typically referred to as cryoablation systems, cryospray systems, cryospray ablation systems, cryosurgery systems, cryosurgery spray systems and/or cryogen spray ablation systems. As typically used, “cryogen” refers to any fluid (e.g., gas, liquefied gas or other fluid known to one of ordinary skill in the art) with a sufficiently low boiling point (i.e., below approximately −153° C.) for therapeutically effective use during a cryogenic surgical procedure. Suitable cryogens may include, for example, liquid argon, liquid nitrogen and liquid helium. Pseudo-cryogens such as liquid carbon dioxide and liquid nitrous oxide that have a boiling temperature above −153° C. but still very low (e.g., −89° C. for liquid N₂O) may also be used.

During operation of a cryospray ablation system, a medical professional (e.g., clinician, technician, physician, surgeon, etc.) directs a cryogen spray onto the surface of a treatment region via a cryogen delivery catheter. The medical professional may target the cryogen spray visually through a video-assisted device or scope, such as a bronchoscope, endoscope, colonoscope or ureteroscope. Cryogen spray exits the cryogen delivery catheter at a temperature ranging from 0° C. to −196° C., causing the target tissue to freeze or “cryofrost.” As liquid cryogen exits the cryogen delivery catheter and impacts upon the target, it converts to a gaseous state with a significant increase in volume. For example, 1 cubic centimeter (cm³) of liquid nitrogen converts to 694 cm³ of nitrogen gas at body temperature. If not properly isolated and/or vented from the patient and allowed to progress further into the body from the treatment site, these expanding gases may cause undue distention and may have life-threatening consequences, including, for example, pneumothorax of the lungs and perforations of the upper or lower gastrointestinal (GI) tract.

Accordingly, various advantages may be realized by devices and methods as disclosed herein which isolate a treatment region within the body from other regions, such as isolating the esophagus and gastroesophageal junction from the stomach to prevent or significantly inhibit gas from accumulating and progressing distally beyond the treatment region.

### SUMMARY

The present disclosure in various embodiments includes a barrier member at the end of a shaft for isolating a treatment region in the body from other regions. The barrier member may isolate a proximal region of the digestive tract from a distal region of the digestive tract such that fluids introduced during the treatment do not substantially communicate between the regions. Medical devices and procedures may treat a region without disrupting another region.

In one aspect, a device for use in a lumen of patient's body to isolate a proximal region of the lumen from a distal region of the lumen may include an elongate shaft having a proximal end, a distal end, and a length along a longitudinal axis dimensioned to reach from outside of the body to a barrier position within the lumen between the proximal region and the distal region. The shaft may include a supply lumen and a suction lumen extending along the longitudinal axis of the shaft. A barrier member may be at a distal end of the shaft. An expandable chamber may extend around an outer edge of the barrier member. A supply line may be at the distal end of the shaft in fluid communication with the supply lumen and the expandable chamber. A suction channel may be disposed about a proximal portion of the expandable chamber. A suction line may be at the distal end of the shaft in fluid communication with the suction lumen and the suction channel. A fluid may be conveyed from the supply lumen through the supply line inflating the expandable chamber. The fluid may be a gas selected from air, oxygen, nitrogen, and carbon dioxide. A vacuum pressure may be applied from the suction lumen through the suction line creating a suction along the suction channel. The barrier member may have a delivery configuration and a deployed configuration. The device may include an elongate delivery sheath having a lumen configured to slidingly receive the shaft therealong. The barrier member may be transitionable from the deployed configuration to the delivery configuration. The barrier member may be configured to transition from the delivery configuration to the deployed configuration with the expandable chamber as it expands to engage a surrounding wall of the lumen. The suction channel may be configured to sealingly engage the device against the surrounding wall of the lumen when the barrier member is in the deployed configuration. The lumen may be the gastrointestinal (GI) tract. The barrier position may be located distal to the gastroesophageal junction. The proximal region may be the esophagus and the distal region may be the stomach. The barrier position may be located in a fundal region of the stomach. The suction channel may be configured to sealingly engage the device in the fundal region. The barrier position may be located distal to the pylorus, in which case the proximal region may be the stomach, and the distal region may be the duodenum. The lumen may be an airway of the lungs, in which case the proximal region may be a lower level airway of the lumen and the distal region may be a higher level airway of the lumen. The barrier position may be located between the proximal region and the distal region. The expandable chamber may be inflatable to a pressure in a range of about 0.5 psi to about 5 psi. The suction channel may be operable with a vacuum pressure applied in a range of about 380 Torr to about 1 Torr. The barrier member may include a compliant material. The compliant material may be selected from the group consisting of a plastic, a urethane, a polymer, and a metallic foil. The length of the elongate shaft may be in the range of about 20 cm to about 120 cm. The barrier member may be constrainable within the sheath in the delivery configuration. The supply line may extend along a proximal surface of the barrier member. The supply line may be an interior gap space formed between a proximal surface and a distal surface of the barrier member. At least one aperture on a proximal portion of the suction channel may be in fluid communication with the suction channel, the suction line, and the suction lumen. At least one aperture may be in fluid communication with an exterior space proximal to the suction channel. The suction channel may include a proximal wall that is an annular and convex surface. The supply lumen and suction lumen may extend coaxially along the shaft with the supply lumen within the suction lumen. The device may include a releasable check valve along either of the supply lumen or supply line. The supply lumen and suction lumen may extend parallel and in contact with each other along the shaft. The barrier member may be biased distally near a center of the barrier and biased proximally near the outer edge. The barrier member may have a concave profile from the center to the outer edge. The expandable chamber may be a toroidal balloon. A portion of a distal surface of the suction channel may include a portion of a proximal portion of the expandable chamber.

In another aspect, a device for use in a lumen of a patient's body to isolate a proximal region of the lumen from a distal region of the lumen may include an elongate shaft having a proximal end, a distal end, and a length along a longitudinal axis dimensioned to reach from outside of the body to a barrier position within the lumen between the proximal region and the distal region. A self-expanding barrier member may be disposed at the distal end of the elongate shaft. A suction channel may be disposed about a proximal portion of the self-expanding barrier. A plurality of apertures may be spaced on a proximal wall of the suction channel around a circumference of the suction channel. The apertures may be in fluid communication with the suction channel. A suction line may be in fluid communication with the suction channel. A suction lumen may be within the elongate shaft and may be in fluid communication with the suction line. The suction channel may be configured to sealingly engage the device against a surrounding wall of the lumen when the self-expanding barrier member is in a deployed configuration. A vacuum pressure may be applied from the suction lumen through the suction line to create suction along the suction channel. The barrier position may be located in a fundal region of the stomach. The suction channel may be configured to sealingly engage the device in the fundal region. The self-expanding barrier member may include a garter spring. The self-expanding barrier member may be circular, substantially flat, and flexible. The self-expanding barrier member may have a delivery configuration and a deployed configuration. The self-expanding barrier member may be configured to transition from the delivery configuration to the deployed configuration along with the expandable chamber the chamber expandable to engage a surrounding wall of the lumen. The expandable chamber may extend around an outer edge of the self-expanding barrier member. A device may include a delivery sheath. The self-expanding barrier member may be configured to transition from the delivery configuration to the deployed configuration when unconstrained from within the sheath. The lumen may be the GI tract. The barrier position may be located distal to the gastroesophageal junction. The proximal region may be the esophagus and the distal region may be the stomach. The circular self-expanding barrier member may have a center and edge, and may be biased distally near the center and biased proximally near the edge. An expandable chamber may extend around an outer edge of the self-expanding barrier member. A supply line may be in fluid communication with the expandable chamber. A supply lumen may extend along the elongate shaft in fluid communication with the supply line.

In another aspect, a method for isolating a proximal region of a lumen from a distal region of the lumen within a patient's body may include inserting an elongate shaft into a body of the patient to a barrier position between the proximal region and distal region of the lumen. The shaft may have a proximal end and a length extending along a longitudinal axis to a barrier member at a distal end of the shaft. The barrier member may have a delivery configuration and deployed configuration. The barrier member may transition from the delivery to the deployed configuration. An outer edge of the barrier member may engage with a surrounding wall of the lumen, such that the barrier member may isolate the distal region from the proximal region at the barrier position. A fluid may be supplied to an expandable chamber at an edge of the barrier member to inflate the chamber and deploy the barrier member. A negative pressure may be created in a suction channel that is disposed on a proximal edge of the barrier member to sealingly engage the barrier member within the lumen at the barrier position. A cryogen fluid may be applied to a wall of the lumen in the proximal region. The cryogen fluid may be a cryogen gas. The gas may be isolated with the barrier member from the distal region and the gas may be evacuated from the proximal region to outside of the patient's body. The proximal region may be an esophagus. The distal region may be a stomach. The barrier position may be an inner surface of the stomach at the outlet of the gastroesophageal junction. A supply lumen may be within the shaft in fluid communication with a supply line. An inflation fluid may be conveyed from the supply lumen through the supply line to inflate an expandable chamber extending around an outer edge of the barrier member. The inflation fluid may be a gas selected from air, oxygen, nitrogen, and carbon dioxide. The barrier member may transition from the delivery configuration to the deployed configuration with the chamber as it expands to engage the surrounding wall of the lumen. The device may be sealingly engaged against the surrounding wall when the barrier member is in the deployed configuration. The lumen may be the GI tract. The barrier position may be located distal to the gastroesophageal junction. The proximal region may be the esophagus. The distal region may be the stomach. The barrier position may be located in a fundal region of the stomach. The barrier member may be sealingly engaged to the surrounding wall of the lumen in the fundal region. The barrier position may be located distal to the pylorus. The proximal region may be the stomach. The distal region may be the duodenum. The lumen may be an airway of the lungs. The proximal region may be a lower-level airway of the lumen. The distal region may be a higher-level airway of the lumen. The barrier position may be located between the proximal region and the distal region.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a device in position in the upper gastrointestinal tract in accordance with an embodiment of the present disclosure.

FIG. 2A illustrates an isometric view of a device in a deployed configuration in accordance with an embodiment of the present disclosure.

FIG. 2B illustrates a cross-sectional side-view of the device in FIG. 2A in accordance with an embodiment of the present disclosure.

FIG. 3A illustrates a delivery catheter and a device in the upper gastrointestinal tract in accordance with an embodiment of the present disclosure.

FIG. 3B illustrates the device of FIG. 3A being delivered to the stomach in accordance with an embodiment of the present disclosure.

FIG. 3C illustrates a cross-sectional view of a system, including a probe and the device of FIGS. 3A and 3B in position in the upper gastrointestinal tract, in accordance with an embodiment of the present disclosure.

FIG. 3D illustrates the device of FIGS. 3A-3C being removed from the stomach in accordance with an embodiment of the present disclosure.

### DETAILED DESCRIPTION

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to cryotherapy systems for use within the upper and lower gastrointestinal tracts and respiratory system, the various systems and methods may be used in a variety of other body passageways, organs and/or cavities, such as the vascular system, urogenital system, lymphatic system, neurological system and the like. The various embodiments of the present disclosure are not necessarily limited to cryotherapy procedures, but may be employed in other medical procedures in which it is desirable to employ a barrier member to isolate a proximal region of the lumen from a distal region of the lumen.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

As used herein, the term “distal” refers to the end farthest away from the medical professional when introducing a device into a patient, while the term “proximal” refers to the end closest to the medical professional when introducing a device into a patient.

As used herein, the term “expandable” refers to the ability to self-expand or cause to be expanded in diameter from a “collapsed,” “unexpanded” or “deflated” configuration to an “expanded” or “inflated” configuration. As used herein, “diameter” refers to the distance of a straight line extending between two points and does not necessarily indicate a particular shape.

As used herein, the term “passive venting” refers to the unassisted venting of gases from within a body lumen to an external location, through a body lumen and natural orifice or through a ventilation tube passing through the same. As used herein, the term “active venting” refers to the mechanically-assisted venting (e.g., via a suction source) of gases from with a body lumen to an external location (e.g., through a ventilation tube, through an endoscope working channel, or through a working channel of a cryogen delivery catheter or other catheter).

The present disclosure relates generally to medical devices and procedures for isolating a treatment region in the body from other regions, such as a treatment region in the digestive tract from other regions of the digestive tract. In particular, the present disclosure relates to devices and methods for performing a treatment within the upper gastrointestinal tract, esophagus, gastroesophageal junction, and/or stomach that utilizes a barrier member to isolate a proximal region of the digestive tract from a distal region of the digestive tract such that fluids introduced during the treatment do not substantially communicate between the regions.

For example, devices and methods for use with cryotherapy systems may include a barrier device with a barrier member configured to be situated at a barrier position to prevent or significantly inhibit the accumulation and distal progression of materials and/or substances, including, but not limited to, cryospray gases (hereafter referred to as “cryospray”), within the body lumen. Exemplary cryotherapy systems in which the present disclosure may be implemented include, but are not limited to, those systems described commonly owned U.S. Pat. Nos. 9,820,797, 9,301,796, and 9,144,449 and U.S. patent application Ser. Nos. 11/956,890, 12/022,013, 14/012,320 and 14/869,814, each of which are herein incorporated by reference in their entirety.

During cryotherapy, as liquid cryogens are sprayed into the human body for example during spray cryotherapy, phase changes with the cryogen yield significant volumes of gas that must be safely evacuated from the patient to avoid complications such as pneumoperitoneum or perforation. This may be accomplished by inserting a conduit and applying a negative pressure to the conduit in order to draw out the fluids from areas susceptible to trapping cryogen gas, for example the stomach. Alternatively, delivering a distal blocking balloon to isolate an area of the lower esophagus or displace a volume of the stomach to prevent distal progression of the expanding cryogen gas may complement or eliminate the need for a suction conduit.

In various embodiments of the present disclosure, a proximal region of a lumen within a patient may be isolated from a distal region, such as within the gastrointestinal tract, to prevent or significantly inhibit unnecessary displacement of gas within the distal region. Such embodiments may isolate the distal region by using suction rather than radial forces, thereby minimizing the risk of stretching or rupture of the lumen, and may provide a more effective seal. Such embodiments may also allow better access to the full esophageal lumen, gastroesophageal junction, and perhaps the proximal portion of the stomach, depending on the desired barrier position.

In various embodiments, a device with a barrier member located in a barrier position may isolate a proximal region of a lumen of patient's body from a distal region. A barrier position may be located, e.g., distal to the gastro-esophageal junction (GEJ). A proximal region may be the esophagus and the distal region may be the stomach. Alternatively, a barrier position may be located in a fundal region of the stomach to allow improved access to the esophagus and GEJ, while a suction channel of a device may be configured to sealingly engage the device in the fundal region to isolate the distal region of the lumen of the GI tract (including the rest of the stomach). A barrier position may also be distal to the pylorus, with a proximal region for treatment may be the stomach, and a distal region that is isolated may be the duodenum. As a further example, a lumen may be an airway of the lungs, a proximal region may be a lower level airway of the lumen, a distal region may be a higher level airway of the lumen, and a barrier position for the device may be at a location between the proximal treatment region and the isolated distal region.

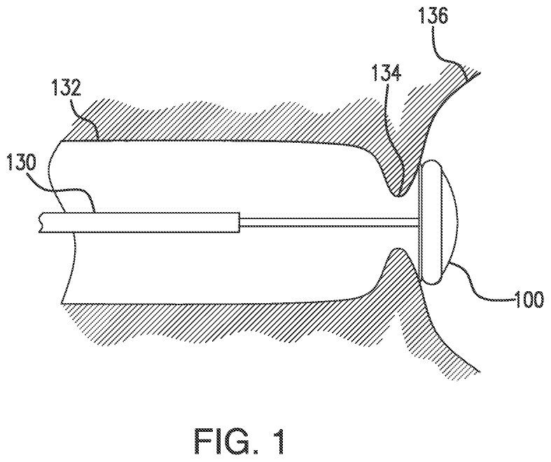

Referring to FIG. 1, an embodiment of a device is illustrated for use in a lumen of a patient's body to isolate a proximal region of a lumen (e.g., GI tract) from a distal region of the lumen. The device of FIG. 1 includes a sheath 130 (e.g., a delivery device or catheter) with the shaft and barrier member of the device within the sheath for insertion into the esophagus to a barrier position for deployment of the barrier member of the device 100. The sheath 130 may be an elongate delivery sheath having a lumen that is configured to slidingly receive an elongate shaft of the device therealong. The barrier member may be constrainable within the sheath 130 in a delivery configuration. The device 100 may be deployed as shown, sealingly engaged to the surrounding wall in a barrier position in the fundus of the stomach to allow for a medical treatment in the proximal region of the lumen with the esophagus and GEJ 134, and into a proximal portion of a stomach 136. The device 100 is shown isolating the esophagus 132 and the gastroesophageal junction 134 from most of the remainder of the stomach 136 and the rest of the GI tract, as the region of the lumen proximal to the barrier member.

Embodiments of the present disclosure may isolate a proximal region of a body lumen of a patient from a distal region within the lumen. The region between the proximal region and the distal region may be a barrier position at which a device with a barrier member is located. With a device located at a barrier position, a medical treatment may be performed within the proximal region, while fluids and/or materials are substantially isolated by the barrier member of the device from moving distally in the lumen beyond the barrier position. The barrier position may be maintained by a barrier member engaging a surrounding wall of the lumen. A barrier member may expandable or self-expanding to engage the wall of the lumen. A barrier member may expand and engage the lumen via an expandable chamber extending around an outer edge of the barrier member. A barrier member may be engaged against the wall of a lumen via suction applied from a suction lumen and suction line to a suction channel that may be disposed about a proximal portion of an expandable chamber. Alternatively, the barrier member may be deployed and engaged with the lumen wall using the expandable chamber and no suction. Contact between the expandable member and the wall of a lumen may be accomplished mechanically with a spring device that is connected between the barrier member and the sheath. The spring may pull the barrier member proximally and/or radially such that the expandable member may engage the wall of the lumen. Alternatively, a line may be connected to the barrier member and extend outside of the patient such that a user may control the line to provide tension upon the barrier member, sealingly engaging the expandable member against a wall of the lumen.

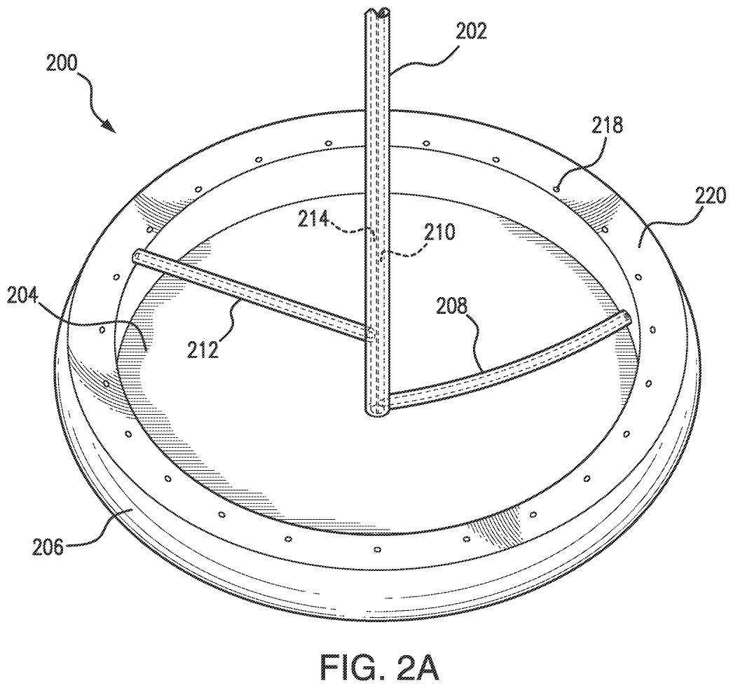

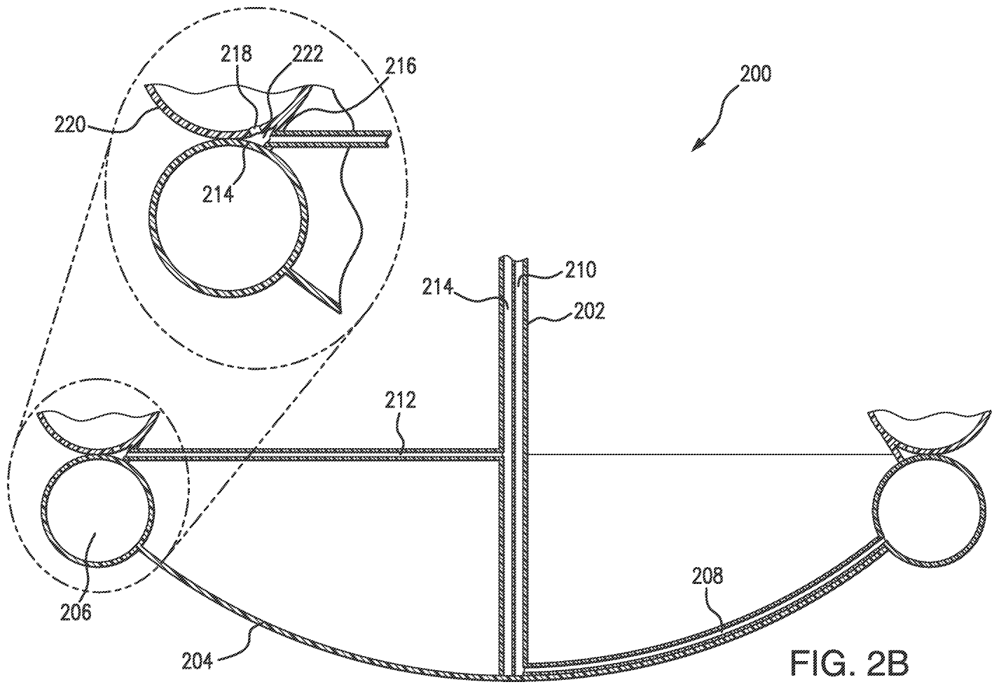

Referring to FIG. 2A, an embodiment of the present disclosure includes a barrier device with an elongate shaft 202 having a proximal end, a distal end, and a length along a longitudinal axis dimensioned to reach from outside of the body to a barrier position. The shaft 202 includes a supply lumen 210 and a suction lumen 214 extending along the longitudinal axis of the shaft 202 with a barrier member 204 at a distal end of the elongate shaft 202. The barrier member 204 illustrated in FIGS. 2A and 2B is in a deployed configuration. An expandable chamber 206 extends around the outer edge of the barrier member 204. The expandable chamber 206 is toroidal in shape about the circumference of the barrier member 204. A supply line 208 at the distal end of the shaft 202 is in fluid communication with the expandable chamber 206. The expandable chamber 206 is disposed on a proximal portion of the barrier member 204. The supply line 208 is in fluid communication with the supply lumen 210 within the elongate shaft 202. In order to expand the expandable chamber 206, the supply lumen 210 is supplied with a fluid that feeds into the supply line 208 and into the expandable chamber 206.

With further reference to FIGS. 2A and 2B, the device includes a suction channel 222. The suction channel 222 is disposed about a proximal portion of the expandable chamber 206. The suction channel 222 is bounded by a proximal channel wall 220 in the proximal direction, by a proximal surface 214 of the expandable chamber 206 in the distal direction, and a radial channel wall 216 in a radial direction towards the longitudinal axis of the elongate shaft 202. The radial channel wall 216 is connected to the proximal channel wall 220 and the proximal surface 214 of the expandable chamber 206. A suction line 212 is in fluid communication with the suction channel 222. The suction line 212 is at the distal end of the shaft 202 and is in fluid communication with a suction lumen 214 within the elongate shaft 202. A series of apertures 218 are spaced on the proximal channel wall 220 around the circumference of the annular suction channel 222, in fluid communication with the suction channel 222, and in fluid communication with an exterior space proximal to the proximal channel wall 220. The apertures 218 are also in fluid communication with the suction line 212 and the suction lumen 214. In order to create a vacuum in the suction channel 222 and thus a suction force along the proximal surface of the proximal channel wall 220, a vacuum source (not shown) is connected to the suction lumen 214. The vacuum source applies a vacuum (negative) pressure on the suction lumen, which in turn creates suction in the suction line 212 and the suction channel 222.

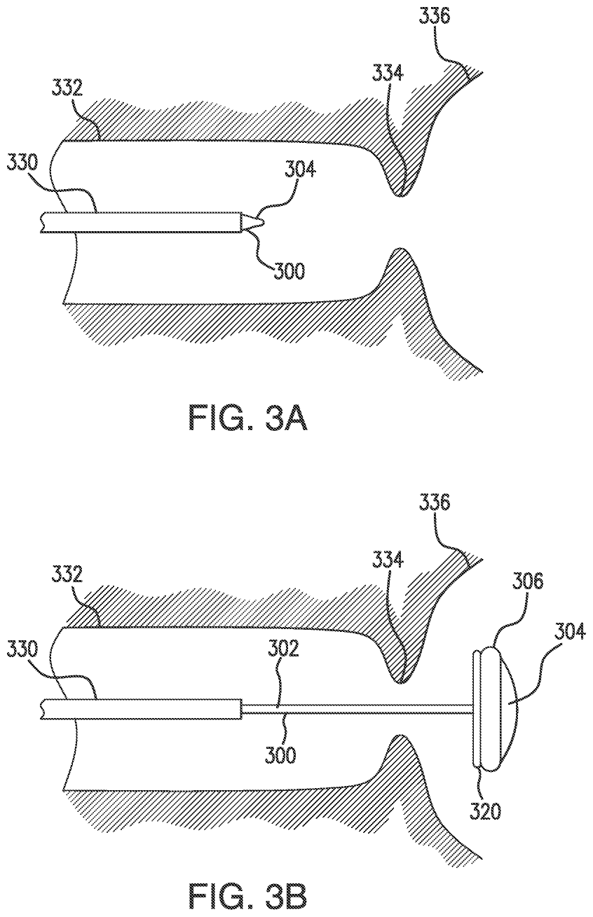

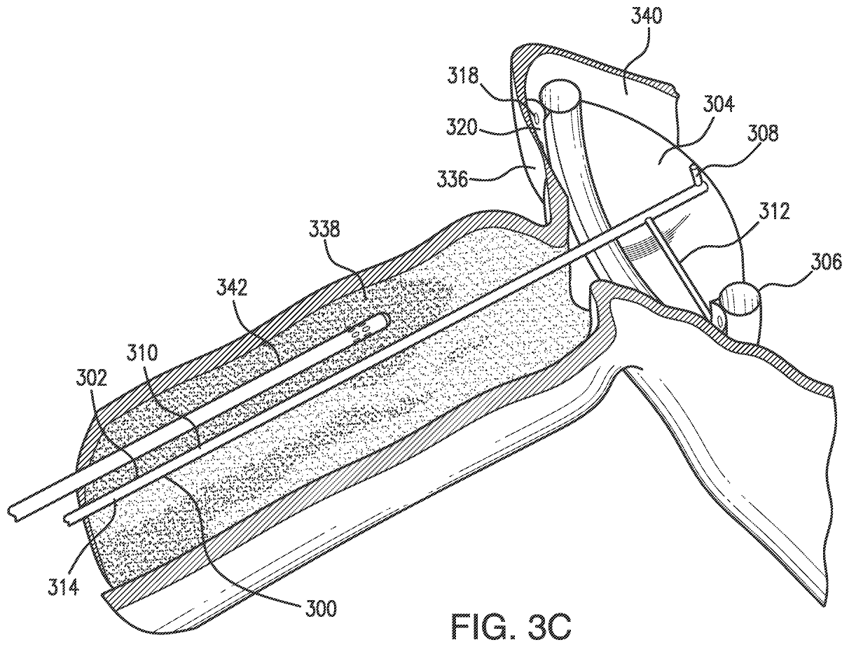

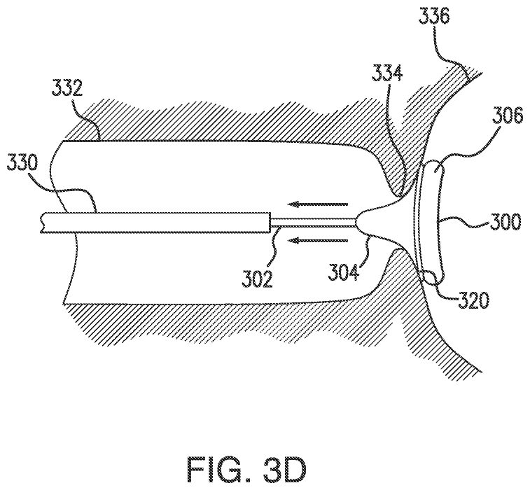

With reference to FIGS. 3A through 3D, an embodiment of a device is depicted inserted within the esophagus 332 via a sheath 330. The sheath 330 is loaded with a device 300 of the present disclosure. With the sheath 300 distal to a target region 338, the device 300 is deployed distally from the sheath 300. In FIG. 3A, the barrier member 304 of the device 300 is in a delivery configuration with the center of a distal surface of the barrier member 304 exposed within the esophagus 332.

Referring to FIGS. 3B and 3C, device 300 is extended out of the sheath 330, into the esophagus 332, distally past the gastroesophageal junction 334, and into the proximal portion of the stomach 336. The device 300, barrier member 304, and expandable chamber 306 are all in a deployed configuration. The supply lumen 310 within the elongate shaft 302 is supplied with a fluid to fill the expandable chamber 306, deploying the barrier member 304 and expandable chamber 306 into the deployed configuration.

Referring to FIG. 3C, a system with device 300 in the deployed configuration and a cryospray catheter 342 in the proximal treatment region of the esophagus 332 is shown. The device is in the barrier position such that the barrier member 304 is between the target region 338 proximal to the barrier member 304 and the isolated region distal to the barrier member 304. The barrier member 304 is also distal to the gastroesophageal junction 334. A suction source may be connected to the suction lumen 314 within the elongate shaft 302 to evacuate the suction lumen 314, suction line 312, and suction channel in order to create a suction force through the apertures 318 along the proximal surface of the proximal channel wall 320 of the suction channel. With the proximal surface of the proximal channel wall 320 brought into proximity of the fundus region of the stomach 336, the suction force in combination with the expansive force of the supplied fluid in the expandable chamber 306 sealingly engages the device 300 against the stomach wall 336, isolating a proximal region that is proximal to the barrier member 304 from a distal region that is distal to the barrier member 304 at a barrier position where the device 300 is located. The apertures 318 within the proximally concave shape of the proximal channel wall 320 assist in improving the suction force. With the suction channel evacuated and positioned into contact with a tissue (e.g., the wall of the lumen in the fundus region of the upper stomach 336), a differential positive pressure seal around the perimeter of the device 300 outside of the proximal channel wall 320 is created. With the device 300 in this position, the esophagus 332, target tissue region 338, esophageal junction 334, and upper wall of the stomach 336 comprise a proximal region that is isolated from the stomach remainder 340. The medical instrument 342 (e.g., a cryospray probe) may be inserted into the esophagus 332 to treat the target region 338 within the proximal region that is proximal to the barrier member 304, while preventing or substantially inhibiting fluids (e.g., cryogen gas) or other particulates from entering the stomach remainder 340 that is distally past the barrier member 304.

Referring to FIG. 3D, after a medical treatment using an embodiment of the present disclosure, the medical instrument 342 is withdrawn from the esophagus 332 and out of the patient. To withdraw the device 300 from the patient, any suction applied is removed, any inflation of the expandable chamber 306 is removed, in order to allow the barrier member 304 to disengage from surrounding wall of the lumen and collapse or be collapsed from the deployed configuration to the delivery configuration. In the delivery configuration or partial delivery configuration, the elongate shaft 302 is translated proximally. This will pull the center of the barrier member 304 proximally while the expandable chamber 306 and proximal channel wall 320 resist proximal movement against the gastroesophageal junction 334. The barrier member 304 inverts from being distal to the expandable chamber 306 to being proximal to the expandable chamber 306. With additional proximal force on the elongate shaft 302, possibly in combination with further transitioning the device from the deployed configuration to the delivery configuration, the device 300 is removed proximally past the gastroesophageal junction 334 into the esophagus 332. The device 300 may continue to be translated proximally into the sheath 330 while the barrier member 304 is in its inverted state. Alternatively, the expandable chamber 306 may be further evacuated prior to proximal translation of the elongate shaft 302. By connecting a suction source to the supply lumen within the elongate shaft 302, the expandable chamber 306 may be evacuated resulting in the device 300 transitioning into the delivery configuration illustrated in FIG. 3A. The elongate shaft 302 may then be translated proximally to withdraw the device 300 past the gastroesophageal junction 334 and into the sheath 330.

Referring further to FIGS. 3A-3D, the target treatment region 338 does not need to be within the esophagus 332. The gastroesophageal junction 334 and upper stomach are also isolated from the stomach remainder 340 with the device in the deployed configuration in this particular barrier position, and can be treated. A medical instrument 342 (e.g., cryospray probe) may be extended distally past the gastroesophageal junction 334 and into the upper portion of the stomach 336 in order to treat the gastroesophageal junction 334 and/or stomach 336.

In various embodiments, described here or otherwise, within the scope of the present disclosure, the barrier member may be an expandable barrier member. The barrier member may be self-expanding. The barrier member may contain a frame with a spring constant that allows it to collapse when drawn into a tube, sheath, catheter, or endoscope. A device delivered through a sheath may have a self-expanding barrier member that transitions from a delivery configuration to a deployed configuration when unconstrained from within the sheath. A garter spring may be connected to the barrier member to assist with its expansion and/or contraction. The barrier member may be a thin diaphragm that may be biased distally near its center and biased proximally at its outer edge. The barrier member may be substantially circular, substantially flat, and/or flexible. The barrier member may have a concave profile from the center to the outer edge. The barrier member may be transitionable from a deployed configuration to a delivery configuration, and the reverse. The barrier member may be configured to transition from the delivery configuration to the deployed configuration as an expandable chamber is inflated or otherwise expanded to engage a surrounding wall of the lumen. The barrier member may be a thin polymeric membrane. The barrier member may comprise a compliant material, a polymer, a urethane, a braid, a mesh, a frame, and/or a metallic foil. A coating may partially or completely cover a barrier member. The coating may be configured to substantially block fluids from advancing distally. The coating may substantially fill-in gaps in the barrier member to form a continuous fluid barrier. The coating may be a flexible elastomeric coating to allow for deformation when the barrier member transitions between the delivery configuration and the deployed configuration. The coating may be applied while the expandable member is in the deployed configuration such that the barrier member is formed into a shape, e.g., a generally frustum-shape, funnel-shape, concave shape, or the like. The coating may increase the stiffness of the barrier member such that the shape and radial stability of the barrier member is reinforced by the coating. The coating may comprise a variety of materials such as, e.g., a urethane, a molded thermoplastic, a thermoplastic urethane, a thermosetting urethane, Pebax, a thermoplastic elastomer, or the like. The barrier member may be made up of two layers with an interior gap space formed between a proximal surface and a distal surface of the barrier member comprising the supply line in fluid communication with the expandable chamber. A tether line may be connected to the barrier member, expandable chamber, the proximal channel wall, and/or the radial channel wall. The tether may be manipulated to transition the device into the deployed and delivery configurations. The tether may also assist to manipulate the proximal channel wall into proximity of the body lumen wall.

In various embodiments, the expandable chamber may be annular. Varying diameters of the expandable chamber will dictate the size of the barrier member and the width of area that can be isolated. A larger diameter of the expandable chamber allows for a wider body lumen that may be engaged by the device. Some embodiments may not include an expandable chamber and rely solely on the barrier member and/or the suction channel to isolate. Such barrier members may include a stiff spring constant to maintain the engagement. The expandable chamber may be replaced by another expanding mechanism such as a garter spring.

In various embodiments, the proximal channel wall may take on various geometries. The proximal surface of the proximal channel wall may be concave in the proximal direction. A radial channel wall of the suction channel may be angled and may reinforce the seal created by the proximal channel and/or the engagement of the device with the wall of a body lumen.

In various embodiments, a supply lumen and a suction lumen may extend parallel and may be in contact with each other along an elongate shaft. A supply lumen and a suction lumen may extend coaxially along the shaft with the supply lumen within the suction lumen. A fluid may be conveyed from a supply lumen through a supply line that inflates an expandable chamber. A fluid may be a gas such as air, oxygen, nitrogen, or carbon dioxide. A vacuum pressure may be applied from the suction lumen through the suction line that creates suction along a suction channel. A releasable check valve may be included along either of the supply lumen or supply line.

In various embodiments, the device may be delivered by a catheter, an endoscope, a retractable sheath, and/or a guide wire. A device may include an elongate shaft with a guide wire lumen that aligns with an aperture at or near the center of the barrier member and extends past the barrier member to allow for the guidewire to guide the device within a patient during insertion and deployment of the barrier member. A length of a shaft may be about 20 cm to about 120 cm.

In various embodiments, the flow of supply fluid between the external fluid source and the supply lumen may be performed manually using, e.g., a syringe, or automatically using an external system. The syringe (or external system) may include a pressure gauge configured to allow a medical professional to confirm that the expandable chamber is sufficiently inflated to contact opposing walls of a body lumen without over-expansion, in order to prevent or significantly inhibit the distal progression of fluids, such as cryospray, and/or sufficiently unexpanded for safe removal from (or repositioning within) the body lumen. For example, an automatically operated external system may include a pressure sensor configured to prevent the delivery of cryogen if the expandable chamber is either unexpanded or insufficiently expanded to establish proper contact of the barrier member with the tissue walls of the body lumen. The expandable chamber may be inflatable to a pressure in a range of about 0.5 psi to about 5 psi. A similar external system may monitor a vacuum pressure of the suction channel to ensure the suction seal is secure and desired level of suction is maintained throughout the procedure. The suction channel may be operable with a vacuum pressure applied in a range of about 380 Torr to about 1 Torr. The expandable chamber and/or the suction channel may include one or more sensors (e.g., pressure sensors, temperature sensors, etc.) to allow the temperature and/or pressure of the expandable chamber to be monitored throughout the cryotherapy procedure. For example, one or more pressure sensors on an inner surface of the expandable chamber may allow the medical professional to introduce or remove supply fluid until a desired level of expansion (e.g., internal pressure) is achieved. In addition, or alternatively, one or more pressure sensors on an outer surface of the expandable chamber may allow the medical professional to monitor the pressure exerted by the expandable chamber against opposing walls of the body lumen (e.g., external pressure). The medical professional may adjust (e.g., increase or decrease) the expanded/unexpanded level as necessary to maintain desired contact between the expandable chamber and body lumen without causing trauma to the body lumen and patient. In one embodiment, the sensors may be configured to wirelessly transmit the pressure and/or temperature measurements such that the medical professional may monitor the suction channel and/or the expandable chamber. For example, if the pressure within the expandable chamber or the level of vacuum pressure on the suction channel decreases below a threshold level during the cryotherapy procedure (e.g., due to leakage of the supply fluid, or condensation of the supply fluid due to proximity to the cryospray, or improper seal against the lumen wall), the medical professional may stop the cryotherapy procedure and reposition or re-expand the expandable chamber and/or suction channel. A system may include an automatic alarm and/or a system shut down function if a readout drops below a threshold level.

In the various embodiments described here and otherwise, devices may be folded, pleated and/or covered by a sheath until deployed to protect the device and facilitate delivery within/through body lumens. Radiopaque materials may be incorporated into or onto the device to allow the location of the barrier member to be visualized with systems capable of detection of radiopaque materials within the patient, such as fluoroscopy imaging.

In various embodiments, a method for isolating a proximal region of a lumen from a distal region of the lumen within a patient's body may include a medical professional inserting an elongate shaft into a body of the patient to a barrier position between the proximal region and distal region of the lumen. The shaft may have a proximal end and a length extending along a longitudinal axis to a barrier member at a distal end of the shaft, the barrier member having a delivery configuration and deployed configuration. The medical professional may transition the barrier member from the delivery configuration to the deployed configuration. The medical professional may engage an outer edge of the barrier member with the surrounding wall of the lumen, such that the barrier member isolates the distal region from the proximal region at the barrier position. The medical professional may create a negative pressure on the proximal side of the barrier member to sealingly engage the barrier member within the lumen at the batter position. A medical professional may supply fluid to an expandable chamber at an edge of the barrier member to inflate the chamber and deploy the barrier member into the deployed configuration. The medical professional may create a negative pressure in a suction channel that is disposed on a proximal edge of the barrier member to sealingly engage the barrier member within the lumen at the barrier position. The medical professional may apply a cryogen fluid to the wall of the lumen in the proximal region. The cryogen fluid may be a cryogen gas. The medical professional may isolate the gas with the barrier member from the distal region and evacuate the gas from the proximal region to the outside of the patient's body.

Any of the embodiments described herein may further benefit from passive or active venting of the treatment area (i.e., proximal to the expandable chamber) through a working channel of an endoscope and/or a working channel of a cryogen delivery catheter. Passive venting may be further facilitated, independent of such vent tubes and/or working channel(s), by managing the body lumen to maintain proper circulation and egress of gases.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A device for use in a lumen of a patient's body to isolate a proximal region of the lumen from a distal region of the lumen, comprising:
an elongate shaft having a proximal end, a distal end, and a length along a longitudinal axis dimensioned to reach from outside of the body to a barrier position within the lumen between the proximal region and the distal region, the shaft including a supply lumen and a suction lumen extending along the longitudinal axis of the shaft;
a barrier member at a distal end of the shaft;
an expandable chamber extending around an outer edge of the barrier member;
a supply line at the distal end of the shaft in fluid communication with the supply lumen and the expandable chamber;
a suction channel disposed about a proximal portion of the expandable chamber; and
a suction line at the distal end of the shaft in fluid communication with the suction lumen and the suction channel.

2. The device of claim 1, wherein the barrier member has a delivery configuration and a deployed configuration.

3. The device of claim 1, wherein the device includes an elongate delivery sheath having a lumen configured to slidingly receive the shaft therealong.

4. The device of claim 2, wherein the barrier member is configured to transition from the delivery configuration to the deployed configuration with the expandable chamber as it expands to engage a surrounding wall of the lumen.

5. The device of claim 4, wherein the suction channel is configured to sealingly engage the device against the surrounding wall of the lumen when the barrier member is in the deployed configuration.

6. The device of claim 1, wherein the supply line extends along a proximal surface of the barrier member.

7. The device of claim 1, wherein the supply line is an interior gap space formed between a proximal surface and a distal surface of the barrier member.

8. The device of claim 1, further comprising:
at least one aperture on a proximal portion of the suction channel in fluid communication with the suction channel, the suction line, and the suction lumen.

9. The device of claim 1, wherein a portion of a distal surface of the suction channel comprises a portion of the proximal portion of the expandable chamber.

10. A device for use in a lumen of a patient's body to isolate a proximal region of the lumen from a distal region of the lumen, comprising:
an elongate shaft having a proximal end, a distal end, and a length along a longitudinal axis dimensioned to reach from outside of the body to a barrier position within the lumen between the proximal region and the distal region; and
a self-expanding barrier member disposed at the distal end of the elongate shaft.

11. The device of claim 10, further comprising:
a suction channel disposed about a proximal portion of the self-expanding barrier;
a plurality of apertures spaced on a proximal wall of the suction channel around a circumference of the suction channel, the apertures in fluid communication with the suction channel;
a suction line in fluid communication with the suction channel; and
a suction lumen within the elongate shaft and in fluid communication with the suction line.

12. The device of claim 11, wherein the suction channel is configured to sealingly engage the device against a surrounding wall of the lumen when the self-expanding barrier member is in a deployed configuration.

13. The device of claim 10, wherein the self-expanding barrier member has a delivery configuration and a deployed configuration.

14. The device of claim 13, wherein the self-expanding barrier member is configured to transition from the delivery configuration to the deployed configuration along with an expandable chamber, the chamber expandable to engage a surrounding wall of the lumen, the expandable chamber extending around an outer edge of the self-expanding barrier member.

15. The device of claim 10, further comprising:
an expandable chamber extending around an outer edge of the self-expanding barrier member;
a supply line in fluid communication with the expandable chamber; and
a supply lumen extending along the elongate shaft in fluid communication with the supply line.

16. A method for isolating a proximal region of a lumen from a distal region of the lumen within a patient's body, comprising:
inserting an elongate shaft into a body of the patient to a barrier position between the proximal region and distal region of the lumen, the shaft having a proximal end and a length extending along a longitudinal axis to a barrier member at a distal end of the shaft, the barrier member having a delivery configuration and deployed configuration;
transitioning the barrier member from the delivery to the deployed configuration; and
engaging an outer edge of the barrier member with a surrounding wall of the lumen, such that the barrier member isolates the distal region from the proximal region at the barrier position.

17. The method of claim 16, further comprising supplying fluid to an expandable chamber at a proximal edge of the barrier member to inflate the chamber and deploy the barrier member.

18. The method of claim 17, further comprising creating a negative pressure in a suction channel that is disposed on the proximal edge of the barrier member to sealingly engage the barrier member within the lumen at the barrier position.

19. The method of claim 16, further comprising applying a cryogen fluid to a wall of the lumen in the proximal region.

20. The method of claim 19, wherein the cryogen fluid is a cryogen gas, and the method further comprises isolating the gas with the barrier member from the distal region and evacuating the gas from the proximal region to outside of the patient's body.
